(19) United States
(12) Patent Application Publication
(10) Pub. No.: US 2017/0145501 A1
(43) Pub. Date: May 25, 2017

(54) APPARATUS AND METHODS OF USING OF BIOMARKERS FOR PREDICTING TNF-INHIBITOR RESPONSE

(71) Applicant: Lasse Folkersen, Copenhagen (DK)

(72) Inventor: Lasse Folkersen, Copenhagen (DK)

(73) Assignee: Lasse Folkersen, Copenhagen (DK)

(21) Appl. No.: 14/947,077

(22) Filed: Nov. 20, 2015

(51) Int. Cl. C12Q 1/68; G06F 19/18; G01N 33/68

(52) U.S. Cl. CPC C12Q 1/6876; G01N 33/6803; G06F 19/18; G01N 2570/00; C12Q 2600/106; G01N 2800/52; C12Q 2600/158

(57) ABSTRACT

Apparatus (10) is provided which is operable to use biomarkers for predicting one or more TNF-inhibitor responses (R). The apparatus (10) includes:
(a) a measuring arrangement (30) for processing one or more biological samples (20) to generate corresponding measurement data (40);
(b) a data processing arrangement (50) for defining a search space including one or more potential biomarkers, wherein the data processor arrangement (50) is operable to obtain biological data derived from a plurality of samples from one or more databases (60);
(c) the data processing arrangement (50) arranged in operation to apply a leave-out-one validation algorithm to the data pertaining to the plurality of samples to obtain probability indications for each search sample related to response to the one or more biomarkers;
(d) the data processing arrangement (50) arranged in operation to identify a subset of samples comprising biomarkers; and
(e) the data processing arrangement (50) arranged in operation.

### TECHNICAL FIELD

The present disclosure relates to apparatus which are operable to employ biomarkers for predicting tumour-necrosis-factor (TNF) inhibitor response, for example the present disclosure relates to apparatus which is operable to determine biomarker predictors, in particular biomarker predictors for TNF-inhibitor response. Moreover, the present disclosure concerns methods of employing aforementioned apparatus to use biomarkers for predicting TNF-inhibitor response, for example to methods of employing aforementioned apparatus for identifying biomarkers for predicting TNF-inhibitor response; the methods enable a patient response to TNF-inhibitors to be profiled. Furthermore, the present disclosure relates to a kit product for profiling according to aforesaid methods; the kit is capable of being used to determine effective medication for patients. Additionally, the present invention relates to a computer program product comprising a non-transitory computer-readable storage medium having computer-readable instructions stored thereon, the computer-readable instructions being executable by a computerized device comprising processing hardware to execute aforesaid methods.

### BACKGROUND

A biological marker, otherwise known as a “biomarker”, is a measureable indictor of a biological state or condition. Such a biomarker is, for example, released into an environment of a living organism as a by-product of life processes of the living organism, or can be present in the organism itself, contained within a body of the living organism. Moreover, such biomarkers comprise, for example, particular compounds, for example in a form of unique chemicals, deoxyribonucleic acid (DNA) from cells and cellular processes, and so forth. The biomarkers are produced in response to various states including life processes, death processes (apoptosis), response to disease, response to therapeutic treatment, response to medication, and so froth. Recent scientific and medical advances have enabled use of biomarkers for medical applications. In such medical applications, biomarkers are now widely used for indicating whether or not some biological state or disease is occurring, and to estimate a severity of such state or disease.

Rheumatoid arthritis (RA) and psoriatic arthritis are conditions to have been investigated using biomarkers. In particular, arthritis studies have been involved with detecting an occurrence of TNF (tumour necrosis factor) in both blood circulation and affected joints. TNF, also known as “TNFα”, is usually present at relatively high concentrations in arthritic patients, and is known to cause synovial inflammation and joint destruction. A normal primary role of TNF in a human body relates to regulating immune cells. Moreover, anti-TNF therapy is frequently used for combating arthritic conditions. Such therapy is known to be effective for only some patients, however. Other forms of known treatment available suffer from a similar limitation, namely is only effective for some individuals. Standard known treatment, therefore, frequently employs a predicted stepped path of events, which first utilises inexpensive and widely available drugs, such as methotrexate, before progressing to other classes of drugs, such as TNF-inhibitors. Examples of TNF-inhibitors include, for example, Etanercept and Adalimumab; “etanercept” and “adalimumab” are trademarks. Such TNF-inhibitors can be relatively expensive, but even these TNF-inhibitors are not guaranteed to be effective for all patients. Further steps of the aforementioned path optionally involve use of next-line drugs, such as Tofacitinib and Tocilizumab; “Tofacitinib” and “Tocilizumab” are trademarks. At a late treatment stage, novel and experimental drugs are optionally used, for example various types of immuno-supressants.

For a patient, the aforementioned path can be temporally long, without a clear indication when beginning the path which treatment will work and which will not. A process of trial and error is time consuming and frustrating for both patient and a corresponding medical practitioner. In addition, using medication for which there is no positive and effective patient response, namely non-response being defined as no beneficial effect of the medication, is a significant waste of medical resources, in terms of both medicine and money.

Unlike established known arrangements for helping patients suffering from certain other diseases, such as cancer, little is done for RA patients to try to predict which drug will be most beneficial for the RA patients. Thus, many RA patients suffer through side-effects of newly initiated therapies without any benefit at all, as well as there being increased expenses to health care systems for non-effective drugs. Effort has therefore been concentrated on developing methods of allowing a prior determination of RA patient suitability for a chosen treatment step. Biomarker use has been under investigation for use in these developing methods.

Known contemporary academic literature contains several descriptions of methods that have been proposed for predicting TNF-inhibitor response. Such methods comprise using serum protein measurements, DNA (deoxyribonucleic acid) genotyping and gene expression levels (via RNA (ribonucleic acid)). In early forms of predictor research, patients who responded positively to anti-TNF treatments and those who did not respond were compared. Important studies were executed based upon DNA-markers, protein levels and gene expression levels. Gene expression is a process by which information from a gene is used when synthesizing a functional gene product. These products may comprise functional RNA. Such a process is used by all known life. In genetics, gene expression occurs at a most fundamental level from which an observable trait can be distinguished.

In the aforementioned academic literature, various types of biomarker studies are also reported. Examples of these studies are elucidated in various patent applications such as WO2014/060785, US2009/0017472 and US2004/0153249. Moreover, these studies comprise using a measurement of, for example, DNA via use of a high-throughput DNA readout method, wherein the measurement of DNA is executed for a subset of biomarkers which exhibit a highest level of prediction and lowest level of redundancy between different biomarkers. Such studies are intended to investigate possible gene and genetics variations, and are based on gene expression.

A published international patent application WO2014/060785 discloses in vitro diagnostic methods of predicting whether or not a patient is likely to be responsive to a treatment employing a TNFα inhibitor. The methods are based on gene expression profiling. By measuring an expression profile of disclosed genes, it is possible to forecast whether a treatment by a TNFα inhibitor will be successful or not.

A published United States patent application US2009/0017472, granted as U.S. Pat. No. 8,092,998, discloses methods of predicting responsiveness to TNFα inhibitors in a subject suffering from an autoimmune disorder, such as rheumatoid arthritis (RA). The methods involve assaying for expression of one or more biomarkers in the subject that are predictive of responsiveness to TNFα inhibitors. A preferred biomarker for such methods is CD11c. The methods optionally further comprise administering a TNFα inhibitor to the subject according to a selected treatment regimen. Kits that include a measuring arrangement for measuring expression of one or more biomarkers that are predictive of responsiveness to TNFα inhibitors for an autoimmune disorder are also provided. Methods of preparing and using databases, and computer program products therefore, for selecting an autoimmune disorder subject for treatment with a TNFα inhibitor are also described.

A published United States patent application US2004/0153249 discloses a method, system and software for screening for, identifing and validating biomarkers that are predictive of a biological state, such as a cell state and/or a patient status.

In a journal “Genomics”, 94 (2009) 423-432, there is provided an article disclosing use of a Convergent Random Forest (CRF) technique methodology for predicting drug response from genome-scale data applied to anti-TNF response and an identification of highly predictive biomarkers. The methodology aims to select from genome-wide expression data a small number of non-redundant biomarkers that could be developed into a simple and robust diagnostic tool. The disclosed method combines a Random Forest classifier and a gene expression clustering to rank and select a small number of predictive genes. Four different data sets were evaluated using the CRF method. The CRF selects a much smaller number of features compared to known alternative, namely five to eight genes, while achieving similar or better performance on both training and independent testing sets of data.

The accuracy and predictive capacity of currently available aforementioned methods fall short of desired performance. A contemporary arbitrary choice of rheumatoid arthritis medication or drugs gives rise to added health care expenses as well as lost time and increased suffering in patients. A specific problem is that it is currently not possible to know a priori which drug will be most beneficial in a given patient, such as patients suffering from RA.

### SUMMARY

The present disclosure seeks to provide an improved method of predicting TNF-inhibitor response, wherein the improved method can be applied to determine a suitability of a given patient for a possible treatment; this method does not constitute a method of treating the animal or human body, excluded from patent right protection,

Moreover, the present disclosure seeks to provide an apparatus for implementing aforementioned methods of predicting TNF-inhibitor response.

Furthermore, the present disclosure seeks to provide an improved method of detecting biomarkers associated with rheumatoid arthritis or inflammatory bowel disease or systemic lupus erythematous or psoriasis.

According to a first aspect, there is provided an apparatus which is operable to use biomarkers for predicting one or more TNF-inhibitor responses (R), characterized in that the apparatus includes:
(a) a measuring arrangement for processing one or more biological samples to generate corresponding measurement data;
(b) a data processing arrangement for defining a search space including one or more potential biomarkers, wherein the data processor arrangement is operable to obtain biological data derived from a plurality of samples from one or more databases;
(c) the data processing arrangement arranged in operation to apply a leave-out-one validation algorithm to the data pertaining to the plurality of samples to obtain probability indications for each search sample related to response to the one or more biomarkers;
(d) the data processing arrangement arranged in operation to identify a subset of samples comprising biomarkers corresponding to a pre-determined regression threshold criterion (P) to obtain a preferred set of biomarkers; and
(e) the data processing arrangement arranged in operation to restrict the search space to known TNF-inhibitor responders when predicting the one or more TNF-inhibitor responses (R).

The invention is of advantage in that it provides an improved apparatus for generating one or more TNF-inhibitor responses (R) by using database data and biological sample data in a more efficient and effective manner.

Optionally, in the apparatus, the data processing arrangement is operable to restrict the search space to 126 predictors as comprised in Appendix A.

Optionally, in the apparatus, the data processing arrangement is operable to restrict the search space to a plurality of biomarkers selected from a literature data-mining algorithm using one or more following keywords: TNF-inhibitor AND rheumatoid arthritis OR inflammatory bowel disease OR systemic lupus erythematous OR psoriasis.

Optionally, in the apparatus, the biological data comprises at least one of:
(i) DNA or RNA or protein data;
(ii) DNA and RNA and protein data.

Optionally, in the apparatus, the data processing arrangement is operable to combine DNA-RNA-protein data to obtain a single prediction signature for use in predicting the one or more TNF-inhibitor responses (R).

Optionally, in the apparatus, the leave-one-out validation comprises use of a random forest algorithm.

Optionally, in the apparatus, the pre-determined regression criterion is less than 0.05. Optionally, in the apparatus, the one or more databases comprise a multi-omics bio-bank, comprising data for at least two of: proteins, genotypes, gene expression.

Optionally, in the apparatus, the search space and the biological data comprise data and potential biomarkers, respectively, that are selected according to one or more medical conditions or a set of medical conditions, wherein the medical conditions are selected for; rheumatoid arthritis OR inflammatory bowel disease OR systemic lupus erythematous OR psoriasis.

Optionally, the apparatus is operable to use a first result for a preferred set of biomarkers, and the apparatus is operable to investigate a patient bio-sample to determine a presence in a bio-sample from the patient of at least one of the preferred set of biomarkers OR at least two of said biomarkers OR two biomarkers originating from different types of -omic data.

Optionally, the apparatus is operable to use a second result for a signal prediction signature, and is operable to investigate a patient bio-sample to determine the presence in the patient bio-sample of the single prediction signature. More optionally, in the apparatus, the prediction signature comprises biomarkers for rheumatoid arthritis. Yet more optionally, in the apparatus, the prediction signature includes a set of six TNF-inhibitor biomarkers comprising a protein ratio of sICAM1/CXCL13 AND genotypes rs12570744, rs2814707, rs3849942 AND gene expression CX3CR1, CYP4F12.

Optionally, the apparatus is operable to determine whether or not a given patient being tested is eligible for treatment for rheumatoid arthritis OR inflammatory bowel disease OR systemic lupus erythematous OR psoriasis.

According to a second aspect, there is provided a method of using an apparatus which is operable to use biomarkers for predicting one or more TNF-inhibitor responses (R), characterized in that the method includes:
(a) using a measuring arrangement for processing one or more biological samples to generate corresponding measurement data;
(b) using a data processing arrangement to define a search space including one or more potential biomarkers, wherein the data processor arrangement is operable to obtain biological data derived from a plurality of samples from one or more databases;
(c) using the data processing arrangement to apply a leave-out-one validation algorithm to the data pertaining to the plurality of samples to obtain probability indications for each search sample related to response to the one or more biomarkers;
(d) using the data processing arrangement to identify a subset of samples comprising biomarkers corresponding to a pre-determined regression threshold criterion (P) to obtain a preferred set of biomarkers; and
(e) using the data processing arrangement to restrict the search space to known TNF-inhibitor responders when predicting the one or more TNF-inhibitor responses (R).

Optionally, the method includes using the data processing arrangement to restrict the search space to 126 predictors as comprised in Appendix A.

Optionally, the method includes using the processing arrangement to restrict the search space to a plurality of biomarkers selected from a literature data-mining algorithm using one or more following keywords: TNF-inhibitor AND rheumatoid arthritis OR inflammatory bowel disease OR systemic lupus erythematous OR psoriasis.

Optionally, in the method, the biological data comprises at least one of:
(i) DNA or RNA or protein data;
(ii) DNA and RNA and protein data.

Optionally, the method includes using the data processing arrangement to combine

DNA-RNA-protein data to obtain a single prediction signature for use in predicting the one or more TNF-inhibitor responses (R).

Optionally, in the method, the leave-one-out validation comprises use of a random forest algorithm.

Optionally, in the method, the pre-determined regression criterion is less than 0.05.

Optionally, in the method, the one or more databases comprise a multi-omics bio-bank, comprising data for at least two of: proteins, genotypes, gene expression.

Optionally, in the method, the search space and the biological data comprise data and potential biomarkers, respectively, that are selected according to one or more medical conditions or a set of medical conditions, wherein the medical conditions are selected for; rheumatoid arthritis OR inflammatory bowel disease OR systemic lupus erythematous OR psoriasis.

Optionally, the method includes using a first result for a preferred set of biomarkers, and investigatin a patient bio-sample to determine a presence in a bio-sample from the patient of at least one of the preferred set of biomarkers OR at least two of said biomarkers OR two biomarkers originating from different types of -omic data.

Optionally, the method includes using a second result for a signal prediction signature,and investigating a patient bio-sample to determine the presence in the patient bio-sample of the single prediction signature. More optionally, in the method, the prediction signature comprises biomarkers for rheumatoid arthritis. Yet more optionally, in the method, the prediction signature includes a set of six TNF-inhibitor biomarkers comprising a protein ratio of sICAM1/CXCL13 AND genotypes rs12570744, rs2814707, rs3849942 AND gene expression CX3CR1, CYP4F12.

Optionally, the method includes determining whether or not a given patient being tested is eligible for treatment for rheumatoid arthritis OR inflammatory bowel disease OR systemic lupus erythematous OR psoriasis.

According to a third aspect, there is provided a kit product for profiling according to the method of the second aspect, wherein the kit product comprises an arrangement for determining a presence in a patient bio-sample of at least one of a preferred set of biomarkers OR at least two of the biomarkers OR two biomarkers originating from different types of -omic data OR a single prediction signature.

Optionally, in the kit product, the single prediction signature comprises biomarkers for rheumatoid arthritis. More optionally, in the kit product, the biomarkers include a set of six TNF-inhibitor biomarkers comprising a protein ratio of sICAM1/CXCL13 AND genotypes : rs12570744, rs2814707, rs3849942 AND gene expression CX3CR1, CYP4F12.

According to a fourth aspect, there is provided a computer program product comprising a non-transitory computer-readable storage medium having computer-readable instructions stored thereon, the computer-readable instructions being executable by a computerized device comprising processing hardware to execute a method pursuant to the aforementioned second aspect.

It will be appreciated that features of the invention are susceptible to being combined in any combination without departing from the scope of the invention as defined by the appended claims.

### DESCRIPTION OF THE DIAGRAMS

Embodiments of the present disclosure will now be described, by way of example only, with reference to the following diagrams wherein:

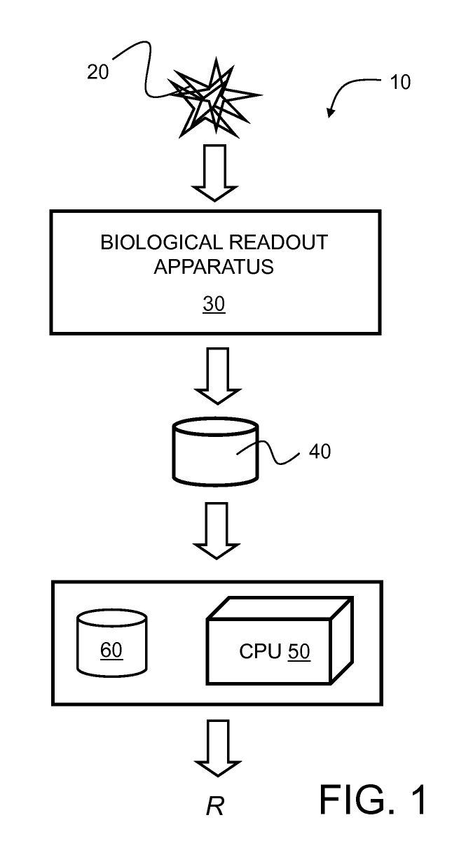

FIG. 1 is a schematic illustration of apparatus according to the present disclosure;

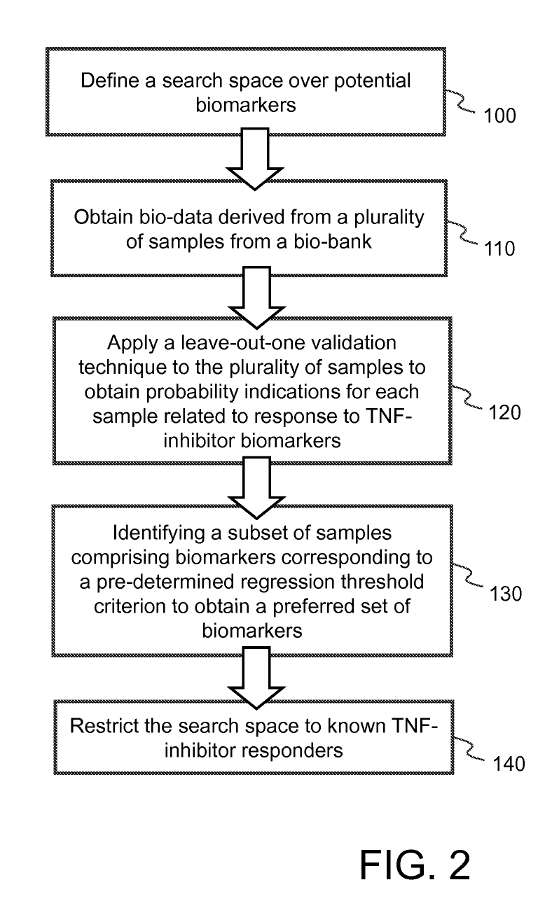

FIG. 2 is an illustration of a method according to the present disclosure for use with the apparatus of FIG. 1; and

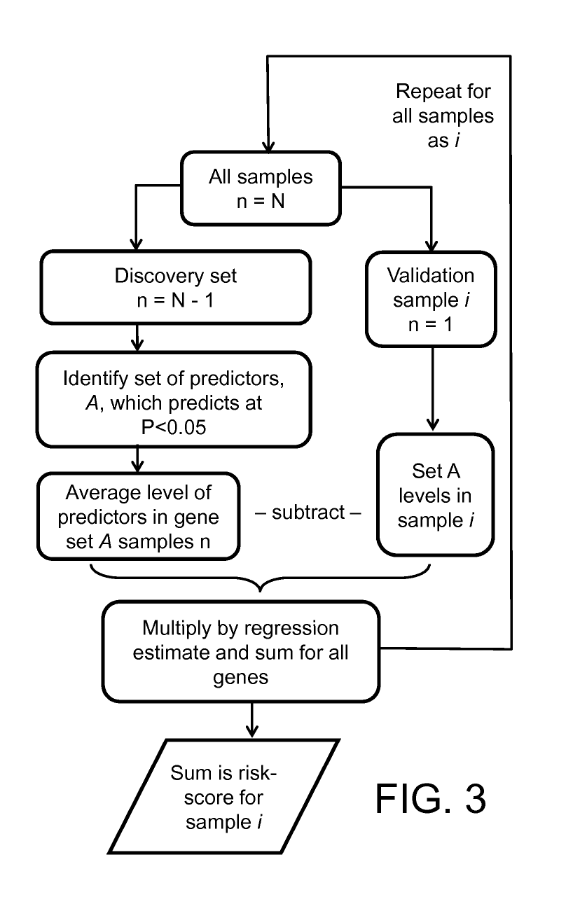

FIG. 3 is an illustration of a leave-one-out algorithm employed in the apparatus of FIG. 1.

In the accompanying diagrams, an underlined number is employed to represent an item over which the underlined number is positioned or an item to which the underlined number is adjacent. A non-underlined number relates to an item identified by a line linking the non-underlined number to the item. When a number is non-underlined and accompanied by an associated arrow, the non-underlined number is used to identify a general item at which the arrow is pointing.

### DESCRIPTION OF EMBODIMENTS OF THE DISCLOSURE

When describing embodiments of the present disclosure, it will be appreciated that specific names may be considered in terms of type; for example, in terms of medications referred to by a tradename or trademark. Moreover, it will be appreciated that there are several drugs grouped under a name “TNF-inhibitors”, for example: Infliximab (Remicade), Adalimumab (Humira), Certolizumab pegol (Cimzia), Golimumab (Simponi), and Etanercept (Enbrel) are specific names that are contemporarily employed. Each medication is produced and marketed by a mutually different pharmaceutical company. However, their function is almost mutually identical and they can be considered as being of one type. Such commercial names should not be considered as limiting to the concepts and implementations of embodiments of the present disclosure.

Referring to FIG. 1, an apparatus pursuant to the present disclosure is indicated generally by 10 and is operable to process a biological sample 20 via a readout apparatus 30, for example a high-throughput DNA reader (for example manufactured by Illumiona Inc. or similar), to generate corresponding measurement data 40, including results indicative of a occurrence of, and a concentration of, one or more biomarkers. Moreover, the apparatus 10 includes a data processing arrangement 50 including at least one data processor (CPU) 50 coupled to one or more databases 60 for processing the measurement data 40 to generate a result “R” which can be used to indicate a particular biological condition pertaining to a given animal or human being from which the biological sample 20 is obtained. In other words, the apparatus 10 is operable to sense a real physical variable, namely biological material composition, and to process corresponding sensing results to generate a useful output, denoted by “R”.

In FIG. 2, there is shown a method according to the present disclosure employed in the apparatus 10 illustrated in FIG. 1. The method includes a first step 100 of defining a search space over potential biomarkers. Moreover, the method includes a second step 110 of obtaining biological data derived from a plurality of samples from a bio-bank; this second step 110 is, for example, provided with the measurement data 40 from the readout apparatus 30 . Furthermore, the method includes a third step 120 of applying a leave-out-one validation technique to the plurality of samples to obtain probability indications for each sample related to response to TNF-inhibitor biomarkers. Addditionally, the method includes a fourth step 130 of identifying a subset of samples comprising biomarkers corresponding to a pre-determined regression threshold criterion to obtain a preferred set of biomarkers. Lastly, the method includes a step 140 of restricting the search space to known TNF-inhibitor responders.

In contradistinction to operation of the apparatus 10, studies comprised in the state of the art, designed to search for prediction biomarkers, are inadequate with regards to a number of biomarkers that are tested, relative to a number of samples available. Such inadequacy result from a multiple testing burden, or sometimes known colloquially as a “winners curse”. Briefly, it means that the more tests are made, the more are positive by chance, namely give rise to “false-positives”. Recognizing such a “false positive” characteristic, the present disclosure aims to provide an improved method of predicting useful biomarkers by a voluntary restriction of a search space employed, so that the method provides an outcome that has a reduced risk of being false-positive. Contents comprised in this restricted search space, according to an embodiment of the present disclosure, are at least partially based upon data-mining of academic literature regarding predicting TNF-responses, but may comprise other inputs of experimental information or practice. For example, such data-mining of academic literature is performed in result of the aforementioned one or more databases 60.

In one optionally example embodiment of the present disclosure, a search space of 126 potential predictors is used at a start of an analysis related to biomarker predictors for optimizing a choice of treatment for rheumatoid arthritis patients. A list of the 126 potential predictors or biomarkers employed for this embodiment is shown in Appendix A that is herewith appended. Formal power-calculations show that this search-space restriction enables more efficient detection of an effect change down to 0.99 TNF-response standard-deviations, in contrast to a full scan of all omics-data which would only be able to detect down to 1.34 standard-deviations. In other words, this pre-definition allows a more sensitive test to be achieved in the apparatus 10.

In the foregoing, the word “omics” informally refers to a field of study in biology ending in “-omics”, such as genomics, proteomics or metabolomics. The related suffix -ome is used to address objects of study of such fields, such as a genome, proteome or a metabolome, respectively. “Omics” aims to achieve a collective characterization and quantification of pools of biological molecules that translate into a structure, a function, and dynamics of an organism or organisms. “Omics” data refers to a collection of data derived from an -omic field, such as aforementioned genomics, which can comprise many mutually different forms or classes of data. In a case of genomics, such data optionally comprises RNA data or gene sequencing information, as well as proteins, genotypes and gene expression. Thus, the term “-omic data” is to be interpreted broadly in scope for purposes of embodiments of the present disclosure.

In an example embodiment of the present disclosure, there is employed a biobank, for example incorporated into the aforementioned one or more databases 60 of the apparatus 10, in which an analysis is performed in operation, wherein the analysis compriss a combined use of DNA, RNA and protein data in search for TNF-response biomarkers. This example embodiment should not be considered as limiting, as other types of -omic data are optionally also used, either in addition to, or as a substitution to, aforesaid DNA, RNA and protein data. Patients, whose biological samples are analyzed and corresponding analysis results comprised in the biobank, are all assumed in the example to be undergoing treatment change or starting a new treatment regimen. The patients are divided into 3 patients groups: one group of patients initiating MTX treatment (cohort A), one group of patients initiating biologics treatment (namely anti-TNF) (cohort B), and one group of patients initiating a second-line biologics treatment, which could be either anti-TNF or other biologics (cohort C). All participants are asked to donate blood samples at a baseline visit (timepoint Om) and at a follow-up visit approximately 3 months later (timepoint 3 m). At both visits, a wide range of clinical scores are evaluated, such as DAS28, CRP, ESR, full medical history, as well as samples for RNA sequencing of PBMC, FACS analysis of whole blood and serum and plasma for protein biomarkers. A total of 492 samples from 246 different individuals are optionally involved, for example, in the example embodiment.

In the foregoing, “DAS28” refers to a DAS28 score, which is a measure of disease activity in rheumatoid arthritis (RA); “DAS” is an abbreviation of “disease activity score”, and the number “28” refers to twenty eight joints that are examined in such an assessment.

Moreover, in the foregoing, “CRP” refers to a cAMP receptor protein which is operable to bind to operon control sequences for affecting an upregulation in gene expression, namely by acting as a transcription factor. Thus, C-reactive protein is a protein measured in blood, that shows increased level when there is inflammation in the body.

Furthermore, “ESR” refers to erythrocyte sedimentation rate which is a measure of settling of red blood cells in a tube of blood within a period of one hour. Additionally, “PBMC” refers to peripheral blood mononuclear cells which provide specific information regarding one or more inflammatory pathways. Thus, “ESR” referes to a subset of cell types commonly found and extracted in blood tests; in a context of the present disclosure, they are of interest because they are a critical component in an immune system of a given person or animal.

Yet additionally, “FACS” refers to fluorescence activated cell sorting, that can be used for isolating specific types of cells for further analysis. Thus, “FACS” is a technology that is employed for sorting and counting cells, typically from a blood sample.

For protein measurements, all citrated plasma samples are investigated using, for example, multiple commercial protein biomarker panels. These included 62 proteins in the HumanMAP assay (Myriad RMB), and 12 proteins in the VectraDA (Crescendo), as well as 33 autoantibodies using an assay technique from Phadia. All plasma protein analysis are usefully performed at specialist biological analysis facilities according to the standard laboratory protocols as indicated by respective manufacturers of the biomarker panels.

For gene expression measurements, RNA is purified from PBMC samples collected using a standard known method of RNA purification. RNA is usefully sequenced, for example, at the proprietary Aros Applied Biotechnology Centre, using a widely available RNA-sequencing protocol named Illumina HiSeq 2000, employing a proprietary TruSeq RNA sample preparation kit and a 2×100bp paired end setup. Samples are usefully randomized to flow cells for obtaining balanced distribution of cohort type and drug response magnitude. A pre-filtering on quality of reads is usefully applied, namely removing adaptors and ensuring high quality of data. Such activity is then usefully followed by alignment to an ensemble GRCh37 genome. Gene expression is usefully quantified with TMM-normalization, mean-scaling and log 2 transformation, which is necessary in order to employ statistical tests as will be described in greater detail later.

For DNA genotyping, DNA is usefully collected in citrated vacutainers and thereafter purified according to a standard proprietary QIAsymphony buffy coat protocol. Purified DNA is then usefully genotyped using a proprietary HumanOmniExpress BeadChip Kit and Illumina OmniExpress arrays (12v1). Altogether, for example, 719665 SNPs are usefully genotyped. Of these, from experimental results, 5633, SNPs may potentially fail a Hardy-Weinberg equilibrium test and are then omitted from analysis. Altogether, 230 individuals are, for example, successfully genotyped via use of one or more microarrays.

In FIG. 3, there is illustratesd a leave-one-out algorithm employed in the aforementioned step 120, and usefully applied to the present example embodiment. The leave-one-out algorithm includes a first analysis of all samples in respect of their DNA, RNA and protein content, because the search comprises a desired combined use of these three potential biomarker types. Thereafter, the leave-one-out algorithm comprises two parallel branches. A first branch includes a leave-one-out subset for providing a discovery set of samples with a subsequent step concerned with identifying a set of biomarker predictors, namely for predicting TNF-inhibitor drug response, with a regression (P) below a given threshold criterion; in this example, the regression P<0.05. The first branch includes a step for determining an average level of predictors in the subset of samples. A second branch includes a step for generating a validation sample for the “one left out” according to the algorithm and for determinating DNA, RNA and protein data for the sample. An iteration step is implemented for computing multiplications for regression coefficients until all samples are considered for the validation sample. Finally, a sum corresponding to a risk score for the validation sample is computed.

A basic selection algorithm setup is illustrated in FIG. 3. Among the 126 potential predictors, most robust predictors are usefully investigated by using this leave-one-out validation as aforementioned. For each sample, there is determined which predictors satisfy P<0.05 in the remaining samples, and those that do are then summarized into a single risk score for the sample that was left out. Such an approach is distinguished by key feature such as the fraction of samples left out, as well as a fact that the algorithm in this example embodiment is based on a well-established known “random forest approach”. Overall, such an approach allows for selection of best predictors from a search space, as well as a calculation of a non-inflated estimate of prediction size.

Although a specific example embodiment of the disclosure is described in the foregoing, it will be appreciated that the embodiment can be optionally modified, for example by using alternative proprietary tests in preparing biological samples for analysis, and subsequently characterizing them. Moreover, the apparatus 10 is susceptible to being implemented at a plurality of geographical locations, wherein parts of the apparatus 10 are linked via a data communication network, for example implemented via use of a combination of biological test laboratories and a data centre including a plurality of computers and associated servers. Results from methods of the disclosure can be printed and/or presented graphically via one or more graphical user interfaces. Yet optionally, the results “R” in FIG. 1 are provided to a machine-based expert system for providing treatment guidance.

Modifications to embodiments of the invention described in the foregoing are possible without departing from the scope of the invention as defined by the accompanying claims. Expressions such as “including”, “comprising”, “incorporating”, “consisting of”, “have”, “is” used to describe and claim the present invention are intended to be construed in a non-exclusive manner, namely allowing for items, components or elements not explicitly described also to be present. Reference to the singular is also to be construed to relate to the plural. Numerals included within parentheses in the accompanying claims are intended to assist understanding of the claims and should not be construed in any way to limit subject matter claimed by these claims.

APPENDIX A
List of 126 TNF-inhibitors defining a search space
in one embodiment of the present disclosure.

| type | id | chromosome | position (MB) |
|---|---|---|---|
| SNP | rs12531738 | 7 | 82.6 |
| SNP | rs17156427 | 7 | 82.6 |
| SNP | rs11980702 | 7 | 82.6 |
| SNP | rs10265155 | 7 | 79.4 |
| SNP | rs10833455 | 11 | 21.1 |
| SNP | rs10833456 | 11 | 21.1 |
| SNP | rs12226573 | 11 | 27 |
| SNP | rs12570744 | 10 | 6.7 |
| SNP | rs1503860 | 1 | 160.5 |
| SNP | rs1532269 | 5 | 32 |
| SNP | rs1568885 | 7 | 13.6 |
| SNP | rs17301249 | 6 | 133.3 |
| SNP | rs1800629 | 6 | 31.6 |
| SNP | rs1800871 | 1 | 206.8 |
| SNP | rs1800896 | 1 | 206.8 |
| SNP | rs1813443 | 11 | 100.1 |
| SNP | rs1980422 | 2 | 203.7 |
| SNP | rs1990099 | 7 | 79.4 |
| SNP | rs2812378 | 9 | 34.7 |
| SNP | rs3761847 | 9 | 120.9 |
| SNP | rs4336372 | 5 | 175.2 |
| SNP | rs4411591 | 18 | 6.6 |
| SNP | rs6427528 | 1 | 160.5 |
| SNP | rs7141276 | 14 | 34.7 |
| SNP | rs7932820 | 11 | 21.1 |
| SNP | rs7933314 | 11 | 27 |
| SNP | rs8009551 | 14 | 34.7 |
| SNP | rs940928 | 2 | 108.9 |
| SNP | rs983332 | 1 | 87.7 |
| SNP | rs928655 | 1 | 89.4 |
| SNP | rs13393173 | 2 | 168.5 |
| SNP | rs437943 | 4 | 35.4 |
| SNP | rs10945919 | 6 | 163.8 |
| SNP | rs854555 | 7 | 95.3 |
| SNP | rs854548 | 7 | 95.3 |
| SNP | rs854547 | 7 | 95.3 |
| SNP | rs7046653 | 9 | 27.5 |
| SNP | rs868856 | 9 | 27.5 |
| SNP | rs774359 | 9 | 27.6 |
| SNP | rs2814707 | 9 | 27.5 |
| SNP | rs3849942 | 9 | 27.5 |
| SNP | rs6028945 | 20 | 40.2 |
| SNP | rs6138150 | 20 | 23.9 |
| SNP | rs6071980 | 20 | 40.2 |
| SNP | rs12081765 | 1 | 165.4 |
| SNP | rs7305646 | 12 | 17.1 |
| SNP | rs4694890 | 4 | 48.2 |
| SNP | rs1350948 | 11 | 23.5 |
| SNP | rs7962316 | 12 | 92 |
| SNP | rs7070180 | 10 | 116.1 |
| SNP | rs1024125 | 2 | 173.8 |
| SNP | rs10739625 | 9 | 123.3 |
| Gene | NFKBIA | 14 | 35.4 |
| Gene | CCL4 | 17 | 36.1 |
| Gene | IL8 | | |
| Gene | IL1B | 2 | 112.8 |
| Gene | TNFAIP3 | 6 | 137.9 |
| Gene | PDE4B | 1 | 65.8 |
| Gene | PPP1R15A | 19 | 48.9 |
| Gene | ADM | 11 | 10.3 |
| Gene | CYP3A4 | 7 | 99.8 |
| Gene | AKAP9 | 7 | 91.9 |
| Gene | THRAP3 | 1 | 36.2 |
| Gene | CXCL5 | 4 | 74 |
| Gene | RPSA | 3 | 39.4 |
| Gene | FBXO5 | 6 | 153 |
| Gene | RASGRP3 | 2 | 33.4 |
| Gene | CIAO1 | 2 | 96.3 |
| Gene | PFKFB4 | 3 | 48.5 |
| Gene | HLA-DPB1 | 6 | 33.1 |
| Gene | RPL35 | 9 | 124.9 |
| Gene | RPS16 | 19 | 39.4 |
| Gene | RPS28 | 19 | 8.3 |
| Gene | PSMB9 | 6 | 32.8 |
| Gene | MUSTN1 | 3 | 52.8 |
| Gene | SORBS3 | 8 | 22.5 |
| Gene | EPS15 | 1 | 51.4 |
| Gene | TBL2 | 7 | 73.6 |
| Gene | PTPN12 | 7 | 77.5 |
| Gene | CYP4F12 | 19 | 15.7 |
| Gene | C19orf70 | 19 | 5.7 |
| Gene | COX7A2L | 2 | 42.3 |
| Gene | ELMOD2 | 4 | 140.5 |
| Gene | MRPL22 | 5 | 154.9 |
| Gene | CDHR5 | 11 | 0.6 |
| Gene | CD46 | 1 | 207.8 |
| Gene | KNG1 | 3 | 186.7 |
| Gene | AADAT | 4 | 170.1 |
| Gene | MX1 | 21 | 41.4 |
| Gene | OAS1 | 12 | 112.9 |
| Gene | OAS2 | 12 | 113 |
| Gene | IFIT3 | 10 | 89.3 |
| Gene | IFIT1 | 10 | 89.4 |
| Gene | MX2 | 21 | 41.4 |
| Gene | EIF2AK2 | 2 | 37.1 |
| Gene | IFI6 | 1 | 27.7 |
| Gene | IFITM1 | 11 | 0.3 |
| Gene | IFITM2 | 11 | 0.3 |
| Gene | HLA-DQA1 | 6 | 32.6 |
| Gene | OASL | 12 | 121 |
| Gene | IGHM | 14 | 105.9 |
| Gene | ZFP36L2 | 2 | 43.2 |
| Gene | SRP9 | 1 | 225.8 |
| Gene | IL2RB | 22 | 37.1 |
| Gene | CX3CR1 | 3 | 39.3 |
| Gene | AP1S2 | X | 15.8 |
| Gene | SH2D1B | 1 | 162.4 |
| Gene | GNLY | 2 | 85.7 |
| Gene | CAMP | 3 | 48.2 |
| Gene | SLC2A3 | 12 | 7.9 |
| Gene | MXD4 | 4 | 2.2 |
| Gene | TLR5 | 1 | 223.1 |
| Gene | PSPH | 7 | 56 |
| Gene | CLGN | 4 | 140.4 |
| Gene | C21orf58 | 21 | 46.3 |
| Gene | TBC1D8 | 2 | 101 |
| Gene | ATP5I | 4 | 0.7 |
| Gene | ANKRD55 | 5 | 56.1 |
| Gene | TMEM141 | 9 | 136.8 |
| Gene | ITGAX | 16 | 31.4 |
| Gene | CD83 | 6 | 14.1 |
| Gene | BCL2A1 | 15 | 80 |
| Protein | ICAM1-protein | 19 | 10.3 |
| Protein | CXCL13-protein | 4 | 77.5 |
| Protein | IL8-protein | 4 | 73.7 |
| Protein | ICAM-CXCL13 ratio-protein | 4/19 | 10.3/77.5 |

1. An apparatus (10) which is operable to use biomarkers for predicting one or more TNF-inhibitor responses (R), characterized in that the apparatus (10) includes:
(a) a measuring arrangement (30) for processing one or more biological samples (20) to generate corresponding measurement data (40);
(b) a data processing arrangement (50) for defining a search space including one or more potential biomarkers, wherein the data processor arrangement (50) is operable to obtain biological data derived from a plurality of samples from one or more databases (60);
(c) the data processing arrangement (50) arranged in operation to apply a leave-out-one validation algorithm to the data pertaining to the plurality of samples to obtain probability indications for each search sample related to response to the one or more biomarkers;
(d) the data processing arrangement (50) arranged in operation to identify a subset of samples comprising biomarkers corresponding to a pre-determined regression threshold criterion (P) to obtain a preferred set of biomarkers; and
(e) the data processing arrangement (50) arranged in operation to restrict the search space to known TNF-inhibitor responders when predicting the one or more TNF-inhibitor responses (R).

2. An apparatus (10) as claimed in claim 1, wherein the data processing arrangement (50) is operable to restrict the search space to 126 predictors as comprised in Appendix A.

3. An apparatus (10) as claimed in claim 1, wherein the data processing arrangement (50) is operable to restrict the search space to a plurality of biomarkers selected from a literature data-mining algorithm using one or more following keywords: TNF-inhibitor AND rheumatoid arthritis OR inflammatory bowel disease OR systemic lupus erythematous OR psoriasis.

4. An apparatus (10) as claimed in claim 1, wherein the biological data comprises at least one of:
(i) DNA or RNA or protein data;
(ii) DNA and RNA and protein data.

5. An apparatus (10) as claimed in claim 1, wherein the data processing arrangement (50) is operable to combine DNA-RNA-protein data to obtain a single prediction signature for use in predicting the one or more TNF-inhibitor responses (R).

6. An apparatus (10) as claimed in claim 1, wherein the leave-one-out validation comprises use of a random forest algorithm.

7. An apparatus (10) as claimed in claim 1, wherein the pre-determined regression criterion is less than 0.05.

8. An apparatus (10) as claimed in claim 1, wherein the one or more databases (60) comprise a multi-omics bio-bank, comprising data for at least two of: proteins, genotypes, gene expression.

9. An apparatus (10) as claimed in claim 1, wherein the search space and the biological data comprise data and potential biomarkers, respectively, that are selected according to one or more medical conditions or a set of medical conditions, wherein the medical conditions are selected for; rheumatoid arthritis OR inflammatory bowel disease OR systemic lupus erythematous OR psoriasis.

10. An apparatus (10) as claimed in claim 1, wherein the apparatus (10) is operable to use a first result for a preferred set of biomarkers, and the apparatus (10) is operable to investigate a patient bio-sample to determine a presence in a bio-sample from the patient of at least one of the preferred set of biomarkers OR at least two of said biomarkers OR two biomarkers originating from different types of -omic data.

11. An apparatus (10) as claimed in claim 1, wherein the apparatus (10) is operable to use a second result for a signal prediction signature, and is operable to investigate a patient bio-sample to determine the presence in the patient bio-sample of the single prediction signature.

12. An apparatus (10) as claimed in claim 11, wherein the prediction signature comprises biomarkers for rheumatoid arthritis.

13. An apparatus (10) as claimed in claim 12, wherein the prediction signature includes a set of six TNF-inhibitor biomarkers comprising a protein ratio of sICAM1/CXCL13 AND genotypes rs12570744, rs2814707, rs3849942 AND gene expression CX3CR1, CYP4F12.

14. An apparatus (10) as claimed in claim 10, wherein the apparatus (10) is operable to determine whether or not a given patient being tested is eligible for treatment for rheumatoid arthritis OR inflammatory bowel disease OR systemic lupus erythematous OR psoriasis.

15. A method of using an apparatus (10) which is operable to use biomarkers for predicting one or more TNF-inhibitor responses (R), characterized in that the method includes:
(a) using a measuring arrangement (30) for processing one or more biological samples (20) to generate corresponding measurement data (40);
(b) using a data processing arrangement (50) to define a search space including one or more potential biomarkers, wherein the data processor arrangement (50) is operable to obtain biological data derived from a plurality of samples from one or more databases (60);
(c) using the data processing arrangement (50) to apply a leave-out-one validation algorithm to the data pertaining to the plurality of samples to obtain probability indications for each search sample related to response to the one or more biomarkers;
(d) using the data processing arrangement (50) to identify a subset of samples comprising biomarkers corresponding to a pre-determined regression threshold criterion (P) to obtain a preferred set of biomarkers; and
(e) using the data processing arrangement (50) to restrict the search space to known TNF-inhibitor responders when predicting the one or more TNF-inhibitor responses (R).

16. A method as claimed in any one of claims 15, wherein the one or more databases (60) comprise a multi-omics bio-bank, comprising data for at least two of:
proteins, genotypes, gene expression.

17. A kit product for profiling according to a method as claimed in claim 15, comprising an arrangement for determining a presence in a patient bio-sample of at least one of a preferred set of biomarkers OR at least two of the biomarkers OR two biomarkers originating from different types of -omic data OR a single prediction signature.

18. A kit product as claimed in claim 17, wherein the single prediction signature comprises biomarkers for rheumatoid arthritis.

19. A kit product as claimed in claim 18, wherein the biomarkers include a set of six TNF-inhibitor biomarkers comprising a protein ratio of sICAM1/CXCL13 AND genotypes : rs12570744, rs2814707, rs3849942 AND gene expression CX3CR1, CYP4F12.

20. A computer program product comprising a non-transitory computer-readable storage medium having computer-readable instructions stored thereon, the computer-readable instructions being executable by a computerized device comprising processing hardware to execute a method as claimed in claim 15.
